(19) United States
(12) Patent Application Publication
(10) Pub. No.: US 2008/0195229 A1
(43) Pub. Date: Aug. 14, 2008

(54) Decellularized pericardial tissue

(76) Inventors: Rodolfo C. Quijano, Laguna Hills, CA (US); Hosheng Tu, Newport Beach, CA (US)

Correspondence Address: HOSHENG TU, 15 RIEZ, NEWPORT BEACH, CA, 92657-0116, US

(21) Appl. No.: 11/704,563

(22) Filed: Feb. 9, 2007

(51) Int. Cl. A61F 2/02

(52) U.S. Cl. 623/23.72

(57) ABSTRACT

The invention discloses a decellularized pericardial tissue via chemical treatment with cholic acid or bile salts as a medical device and process of manufacture.

### FIELD OF THE INVENTION

The present invention generally relates to decellularized pericardial tissue. More particularly, the present invention relates to crosslinked decellularized pericardial tissue as medical devices.

### BACKGROUND OF THE INVENTION

Crosslinking of biological tissue material is often desired for biomedical or medical device applications. For example, the structural framework of pericardial tissue has been extensively used for manufacturing replacement heart valve bioprostheses and other implanted structures, wherein it provides good biocompatibility and strength. However, biomaterials derived from collagenous tissue must be chemically modified and subsequently sterilized before they can be implanted in humans. The fixation, or crosslinking, of collagenous tissue may increase strength and reduces antigenicity and immunogenicity.

Collagen sheets fabricated from reconstituted collagen are also used as wound dressings, providing the advantages of high permeability to water vapor and rapid wound healing. Disadvantages include low tensile strength and easy degradation of collagen by collagenase. Crosslinking of collagen sheets reduces cleavage by collagenase and enhances tensile strength.

Clinically, fixation of biological tissue is used to reduce antigenicity and immunogenicity and prevent enzymatic degradation. Various crosslinking agents have been used for fixation of biological tissue. It is therefore desirable to provide a crosslinking agent suitable for use in biomedical applications that will provide acceptable cytotoxicity and that forms stable and biocompatible crosslinked products.

U.S. Pat. No. 6,608,040 discloses chemical modification of biomedical materials with genipin, a naturally occurring crosslinking agent to fix biological tissue. The cytotoxicity of genipin compared with that of glutaraldehyde was previously studied in vitro using 3T3 fibroblasts, the results demonstrating that genipin is substantially less cytotoxic than glutaraldehyde (Sung H W et al., J Biomater Sci Polymer Edn 1999; 10:63-78). Additionally, the genotoxicity of genipin was tested in vitro using Chinese hamster ovary (CHO-K1) cells, the results evidencing that genipin does not cause clastogenic response in CHO-K1 cells (Tsai C C et al., J Biomed Mater Res 2000; 52:58-65).

As is well known, the human knee comprises an articulation of the femur, the tibia and the patella. The femur and the tibia are maintained in a condition of stable articulation by a number of ligaments of which the principal ones are the anterior and posterior cruciate ligaments and the collateral ligaments. The rupture of the anterior cruciate ligament is commonly encountered as a result of sporting injury or the like. This rupture leads to knee instability and can be a debilitating injury. Though less common, the rupture of the posterior cruciate ligament can be equally disabling.

In the past, polymer or plastic materials have been studied as ligament or tendon replacements. Prosthetic ligament replacements made of carbon fibers and Gore-Tex PTFE materials do not have a long durability. Repeated loading of a prosthetic ligament in a young active patient leads to failure of the ligament. It has been found that it is difficult to provide a tough durable plastic material that is suitable for long-term connective tissue replacement. Plastic material could become infected and difficulties in treating such infections often lead to graft failure.

In accordance with the present invention, decellularized tissue grafts for orthopedic and other surgical applications are provided, which have shown to exhibit many of the desired characteristics important for optimal graft function for bone, tendon, ligament, cartilage, muscle, eye, ear, and cardiovascular as well as urological applications.

In some aspects of the invention, a method for promoting autogenous ingrowth of a biological tissue material is also provided, comprising the provision of a natural tissue, removing cellular material from the natural tissue by cholic acids and/or its derivatives, increasing porosity of the natural tissue, loading an angiogenesis agent or autologous cells into the porosity, and crosslinking the natural tissue with a crosslinking agent, the latter preferably of low toxicity and cytotoxicity level.

### SUMMARY OF THE INVENTION

In general, it is an object of the present invention to provide a biological scaffold configured and adapted for tissue regeneration or tissue engineering. In one embodiment, the process of preparing a biological scaffold comprises steps of removing cellular material and/or lipid from a natural tissue and crosslinking the natural tissue with a crosslinking agent, wherein the scaffold is characterized by reduced antigenicity, reduced immunogenicity and reduced enzymatic degradation upon placement inside or on a patient's body. The “tissue engineering” in this invention may include cell seeding, cell ingrowth and cell proliferation into the scaffold or collagen matrix in vivo or in vitro.

It is another object of the present invention to provide a tendon or ligament graft for use as connective tissue substitution or repair, wherein the graft is formed from a segment of connective tissue protein or collagen, and the segment is decellularized and crosslinked with a crosslinking agent resulting in reasonably acceptable cytotoxicity and reduced enzymatic degradation.

It is a further object of the present invention to provide a method for promoting autogenous ingrowth of damaged or diseased tissue selected from a group consisting of bone, ligaments, tendons, muscle and cartilage, the method comprising a step of surgically or interventionally through minimal skin openings, repairing the damaged or diseased tissue by attachment of a tissue graft, wherein the graft is formed from a segment of connective tissue protein or collagen, the segment being decellularized and crosslinked with a crosslinking agent having acceptable cytotoxicity and reduced enzymatic degradation, and wherein the tissue graft may be loaded with growth factors, bioactive agents, or autologous cells (for example, stem cells).

In some aspects, there is provided a biological tissue material or tissue sheet material comprising a process of removing cellular material and lipid from a natural tissue and crosslinking the natural tissue with a crosslinking agent or with ultraviolet irradiation, the tissue material being characterized by reduced antigenicity, reduced immunogenicity and reduced enzymatic degradation upon placement inside or on a patient's body, wherein porosity of the natural tissue is optionally increased, the increase of porosity being adapted for promoting tissue regeneration. In a preferred embodiment, the natural tissue or tissue sheet material is selected from a group consisting of bovine pericardium, equine pericardium, porcine pericardium, ovine pericardium, caprine pericardium, kangaroo pericardium, fascia lata, dura mater and the like. In still another embodiment, the crosslinked decellularized natural tissue material is loaded with at least one growth factor, at least one bioactive agent, or stem cells.

Some aspects of the invention relate to a method or use of repairing a tissue or organ defect in a patient, comprising: providing a decellularized tissue sheet material having acceptable mechanical strengths; repairing the defect by appropriately placing the tissue material at the defect; and allowing tissue regeneration in the tissue material. In a further embodiment, the tissue sheet material is selected from a group consisting of a bovine pericardium, an equine pericardium, an ovine pericardium, a porcine pericardium, a caprine pericardium, a kangaroo pericardium, fascia lata, dura mater and the like. In another embodiment, the tissue sheet material is crosslinked with a crosslinking agent or with ultraviolet irradiation, wherein the crosslinking agent may be selected from the group consisting of genipin, its analog, derivatives, and combination thereof, epoxy compounds, dialdehyde starch, glutaraldehyde, formaldehyde, dimethyl suberimidate, carbodiimides, succinimidyls, diisocyanates, acyl azide, and combinations thereof.

The method of repairing a tissue or organ defect in a patient further comprises a process of increasing porosity of the decellularized tissue sheet material, the process being selected from a group consisting of an enzyme treatment process, an acid treatment process, a base treatment process, and combinations thereof.

Some aspects of the invention provide a process for the production of a decellularized pericardial patch, sheet or strip (collectively coded as pericardial tissue), comprising: providing a pericardium tissue sheet having cells and extracellular matrix; subjecting the sheet to a solution containing bile acid or bile salts which effect the solubilization of cell membranes of the cells present in the tissue sheet; removing the solubilized cell membranes by flushing the tissue sheet with filtered water; and treating the tissue sheet with a crosslinking agent. In one embodiment, it is provided a decellularized pericardial tissue produced by the process of the present invention. The decellularized pericardial tissue would contain less cellular residues because the solubilized membrane detaches from the surface of the extracellular matrix inside the tissue sheet and is relatively easy to remove for example, by flushing with filtered water.

Some aspects of the invention provide a process for the production of a decellularized tissue or tissue sheet, comprising: providing a tissue having cells and extracellular matrix; subjecting the tissue to a solution containing bile acid or bile salts which effect the solubilization of cell membranes of the cells present in the tissue; removing the solubilized cell membranes by flushing the tissue with filtered water; and treating the tissue with a crosslinking agent.

In one embodiment, the tissue sheet is selected from a group consisting of bovine pericardium, equine pericardium, ovine pericardium, porcine pericardium, caprine pericardium, kangaroo pericardium, fascia lata, and dura mater. In another embodiment, the crosslinking agent is selected from a group consisting of genipin, epoxy compounds, dialdehyde starch, glutaraldehyde, formaldehyde, dimethyl suberimidate, carbodiimides, succinimidyls, diisocyanates, acyl azide, and combinations thereof.

In a further embodiment, the process further comprises increasing porosity of the decellularized pericardial tissue or tissue sheet, wherein the porosity increase is carried out by an enzyme treatment process, an acid treatment process, or a base treatment process.

The process may further comprise dehydrating the decellularized tissue. Alternately, the dehydrating is carried out by soaking the decellularized tissue in glycerol or in glycerol-alcohol mixture (for example, 80% glycerol-20% ethanol). Alternately, the process may further comprise lyophilizing (freeze-drying) the decellularized tissue or tissue patch/sheet in a sterile environment, preferably removing all or substantial amount of the crosslinking agent. Thus, for its use, a reconstitution with specially formulated solutions or simple sterile de-ionized water or saline may suffice to return the material to its flexible, durable, strong, viable state.

Some aspects of the invention provide a process for the preparation of a decellularized tissue sheet or pericardial tissue sheet, comprising: providing a tissue sheet having cells and extracellular matrix; subjecting the sheet to a solution which effects the solubilization of cell membranes of the cells present in the tissue sheet; removing the solubilized cell membranes by flushing the tissue sheet with filtered water or sterile saline; and treating the tissue sheet with a crosslinking agent, wherein the solution preferably contains (or is characterized with) a chemical having a chemical structure with at least two contiguous six-carbon rings shaped in cis-configuration or not coplanar configuration (one example shown as the chemical structure in Formula 1). In one embodiment, it is provided a decellularized tissue sheet or pericardial tissue (that is, in a shape of patch, sheet, or strip) produced by the process of the present invention.

### BRIEF DESCRIPTION OF THE DRAWINGS

Additional objects and features of the present invention will become more apparent and the invention itself will be best understood from the following Detailed Description of Exemplary Embodiments, when read with reference to the accompanying drawings.

FIG. 1 shows a schematic process flow chart for manufacturing a pericardial tissue sheet of the present invention.

### DETAILED DESCRIPTION OF EXEMPLARY EMBODIMENTS

The following detailed description is of the best presently contemplated modes of carrying out the invention. This description is not to be taken in a limiting sense, but is made merely for the purpose of illustrating general principles of embodiments of the invention.

“Tissue engineering” or “tissue regeneration” is meant to refer to cell seeding, cell ingrowth and cell proliferation in the decellularized scaffold or collagen matrix devoid of cellular material in vivo or in vitro. Sometimes tissue engineering is enhanced with an angiogenesis factor.

A “tissue material” refers to a biomedical material of biological tissue origin that might be decellularized and crosslinked to form a medical device. A tissue sheet, such as a pericardial sheet, is in a sub-group of tissue material (including sheet form and non-sheet form).

An “implant” refers to a medical device which is inserted into, or grafted onto, bodily tissue to remain for a period of time, such as an extended-release drug delivery device, tissue valve, tissue valve leaflet, drug-eluting stent, vascular graft, wound healing or skin graft, orthopedic prosthesis, such as bone, ligament, tendon, cartilage, and muscle, a strip such as could be used to suspend a tubular structure (such as a ureter or urethra), a flat structure, a globular or oblate structure to return its originally intended function.

A “scaffold” in this invention is meant to refer to a tissue matrix substantially or completely devoid of cellular material and/or lipid substance. A scaffold may further comprise added structure porosity for cell ingrowth or proliferation.

A “decellularization process” is meant to indicate the process for detaching and removing a substantial portion or all of cellular substance from cellular tissue and/or tissue matrix that contains connective tissue protein/collagen, for example, a pericardial sheet.

“Bioactive agent” in this invention is meant to provide a therapeutic, diagnostic, or prophylactic effect in vivo. Bioactive agent may comprise, but not limited to, synthetic chemicals, biotechnology-derived molecules, herbs, cells, genes, growth factors, health food and/or alternate medicines. In the present invention, the terms “drug” and “bioactive agent” are sometimes used interchangeably.

It is one object of the present invention to provide a decellularized biological scaffold chemically treated with a crosslinking agent that is configured and adapted for tissue regeneration/tissue engineering or other surgical/medical applications. In the region having suitable substrate diffusivity, a decellularized biological tissue material with added porosity and chemically treated by a crosslinking agent enables tissue regeneration, and/or tissue engineering in many biomedical applications.

Membranes and Lipids

Every cell is surrounded by a plasma membrane that creates a compartment where the functions of life can proceed in relative isolation from the outside world. Biological membranes consist primarily of protein and lipids; for example, the myelin sheath membrane consists of about 80% lipid and 20% protein. Two main types of lipids occur in biological membranes: phospholipids and sterols. The bile salts are critically important for the solubilization of lipids in a body. For example, it is known that bile salts emulsify fats in the intestine. The hydrophobic side or surface of the bile salt associates with triacylglycerols to form a complex. These complexes aggregate to form a micelle, with the hydrophilic side of the bile salt facing outward. The micelles (that detached from the surface of the extracellular matrix inside the tissue or tissue sheet) would be relatively easy to remove from the extracellular space in the decellularization process.

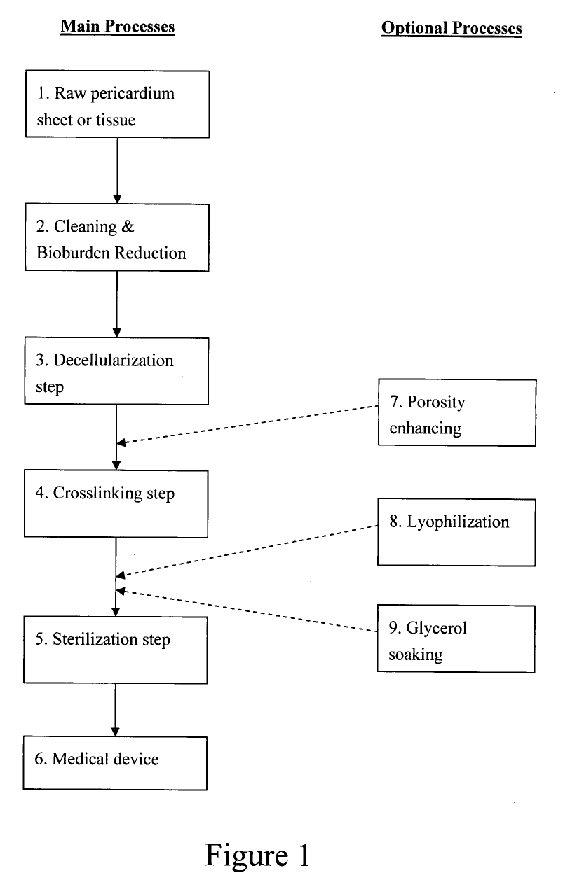

There are currently two mechanisms for tissue sheet or tissue material decellularization. The conventional decellularization process is to increase the differential osmotic pressure across the cellular membrane until the membrane ruptures. It is usually achieved by exposing the cells to a fluid with a lower osmotic pressure, for example, deionized water via a reverse osmosis process. This approach leaves substantial cellular residues or material within the extracellular space still attached/connected to certain internal surface of the tissue sheet. On the contrary, the decellularization approach of the present invention is to delipid or to solubilize lipids (such as the lipids of the membranes), instead of merely breaking up the membranes. The decellularized pericardial sheet would contain less cellular residues because the solubilized membrane detaches from the surface of certain extracellular matrix inside the tissue sheet and is relatively easy to remove since it is already dissociated/detached and free to move around. The majority of the cellular residues having solubilized lipids is much easier to be removed from the extracellular space, for example, by rinsing or flushing with filtered water, sterile saline, sterile alcohol solution or other appropriate solvents. FIG. 1 shows a schematic process flow chart for manufacturing a pericardial tissue sheet of the present invention having main steps of cleaning, bioburden reduction, decellularization, crosslinking, and sterilization, and optional steps of porosity enhancing, lyophilization, and glycerol soaking.

Properties of Cholic Acid

Cholic acid, shown below, has an empirical formula of C₂₄H₄₀O₅.

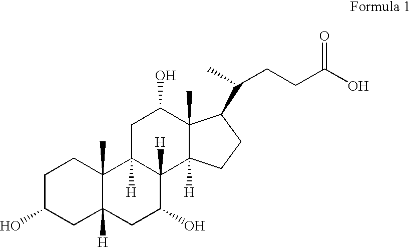

Cholic acid is a bile acid, a white crystalline substance insoluble in water, with a melting point of 200-201° C. Salts of cholic acid (also broadly herein including derivatives of cholic acid) are called cholates or bile salts. Cholic acid is one of the four main acids produced by the liver where it is synthesized from cholesterol. It has active side groups (COOH and OH) and is soluble in alcohol and acetic acid. Cholic acid possess a particular hydrogen (the singular ‘H’ shown at the left lower corner of the structure formula above). As a result, the first six-carbon ring on its right-hand side and the second six-carbon ring on its left-hand side are no longer coplanar but have a cis-configuration (a three-dimension structure). This cis-configuration of two contiguous six-carbon rings improves the detergent properties of the bile acids so they are better able to solubilize lipids.

Glycocholate is an example of a bile salt, derived from glycocholate acid as shown below:

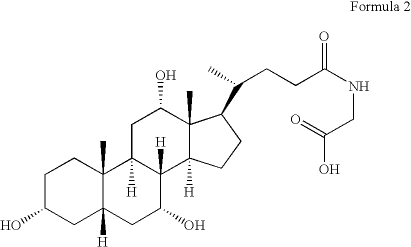

The cholic acid forms a conjugate with taurine, yielding taurocholic acid. Cholic acid and chenodeoxycholic acid are the most important human bile acids. Some other mammals synthesize predominantly deoxycholic acid. The main use of cholic acid is as an intermediate for the production of ursodeoxycholic acid. Ursodeoxycholic acid is a pharmaceutical product that is used for several indications including the dissolution of gallstones and the treatment and prevention of liver disease. Cholic acid (broadly herein defined to include its derivatives) has many different uses in traditional Chinese medicine. Its main use is as an ingredient in the manufacture of artificial calculus bovis (artificial gallstones).

Deoxycholic acid with an empirical formula of C₂₄H₄₀O₄, is shown below:

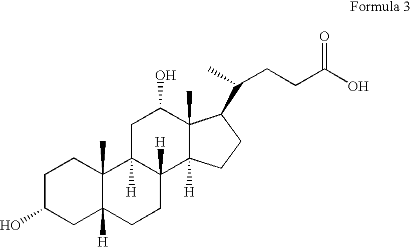

Deoxycholic acid is sparingly soluble in water, but soluble in alcohol and to a lesser extent acetone and glacial acetic acid. Historically deoxycholic acid was used as an intermediate for the production of corticosteroids, which have anti-inflammatory indications.

An emerging use of deoxycholic acid is as a biological detergent to lyse cells and solubilize cellular and membrane components. Some aspects of the invention relate to a process of decellularization of tissue or tissue biomaterial via delipidation as a medical device. It is suggested that cell extraction as a result of cholic acid decellularization removes lipid membranes and membrane-associated antigens as well as soluble proteins. In one embodiment, the process of delipidation or decellularization via delipidation of tissue or tissue biomaterial utilizes cholic acid, deoxycholic acid, or bile salts (including salts of cholic acid and its derivatives, such as glycocholate and deoxycholate) sufficient to delipid and subsequently decellularize the tissue biomaterial.

In a preferred embodiment, the delipidated and/or decellularized tissue or tissue biomaterial is further crosslinked (for example, through ultraviolet irradiation) or treated with a chemical agent, such as genipin, its analog, derivatives, and combination thereof, epoxy compounds, dialdehyde starch, glutaraldehyde, formaldehyde, dimethyl suberimidate, carbodiimides, succinimidyls, diisocyanates, acyl azide, and combinations thereof. Other crosslinking means may also apply to crosslink the decellularized tissue (pericardial and non-pericardial tissues) of the present invention.

Girardot in U.S. Pat. No. 4,976,733, entire contents of which are incorporated herein by reference, discloses a prosthesis having an amount of an anticalcification agent covalently coupled thereto, which anticalcification agent comprises an aliphatic straight-chain or branched-chain, saturated or unsaturated, carboxylic acid or a derivative thereof, which acid contains from about 8 to about 30 carbon atoms, and which acid is substituted with an amino group, a mercapto group, a carboxyl group or a hydroxyl group, which group is covalently coupled to the prosthesis. In one preferred embodiment, the delipidated and/or decellularized tissue or tissue biomaterial is further treated with the herein cited anticalcification agent.

Cholic acid and deoxycholic acid has a low acute toxicity, with LD₅₀ i.v. 50 mg/kg and 15 mg/kg in rabbit, respectively. In general, bile acids and salts have only a minor toxic potential when given by mouth. In large doses, they are likely to have the same effects as saponins; the main action is likely to be irritation of mucous membranes. Parenterally they are much more toxic and may cause hemolysis, a digitalis-like action on the heart and effects on the central nervous system.

Bile is a bitter, yellow to greenish fluid composed of glycine or taurine conjugated bile salts, cholesterol, phospholipid, bilirubin diglucuronide, and electrolytes. It is secreted by the liver and delivered to the duodenum to aid the process of digestion and fat absorption by emulsification of fat products in the upper small intestine. They play role of dissolving cholesterol and accretes into lumps in the gall bladder, forming gallstones. Bile's bicarbonate constituent serves for alkalinizing the intestinal contents. Bile is responsible for as the route of excretion for hemoglobin breakdown products (bilirubin). Excretion of bile salts by liver cells and secretion of bicarbonate rich fluid by ductular cells in response to secretion are the major factors that normally determine the volume of secretion. Bile acids are liver-generated steroid carboxylic acids. Examples of bile acids include cholic acid itself, deoxycholic acid, chenodeoxy colic acid, lithocholic acid, taurodeoxycholate ursodeoxycholic acid, hyodeoxycholic acid and derivatives like glyco-, tauro-, amidopropyl-1-propanesulfonic- and amidopropyl-2-hydroxy-1-propanesulfonic-derivatives of the above bile acids, or N,N-bis (3D Gluconoamidopropyl) deoxycholamide. Salts of bile acids are normally called bile salts.

The primary bile acids (for example, cholic and chenodeoxycholic acid) are conjugated with either glycine or taurine in the form of taurocholic acid and glycocholic acid. The secondary bile acids (deoxycholic, lithocholic, and ursodeoxycholic acid) are formed from the primary bile acids by the action of intestinal bacteria. They are soluble in alcohol and acetic acid. The lithocolyl conjugates are relatively insoluble; excreted mostly in the form of sulfate esters like sulfolithocholylglycine. Most of the bile acids are reabsorbed and returned to the liver via enterohepatic circulation, where, after free acids are reconjugated, they are again excreted.

Sung et al. in U.S. Pat. No. 6,998,418, entire contents of which are incorporated herein by reference, discloses a biological tissue configured and adapted for tissue regeneration, the tissue being characterized by reduced antigenicity reduced immunogenicity and reduced enzymatic degradation upon placement inside a patient's bode with porosity being increased by at least 5%, further comprising an angiogenesis agent, stem cells or autologous cells. Further, the biological tissue may be a bovine pericardium, an equine pericardium, or a porcine pericardium with increasing porosity of the tissue that is provided by an enzyme treatment process, by an acid treatment process, or by a base treatment process. However, the U.S. Pat. No. 6,998,418 patent does not teach the process of delipidation and/or decellularization of tissue biomaterial by utilizing cholic acid (bile acid) or bile salts.

Noishiki et al. in U.S. Pat. No. 4,806,595 discloses a tissue treatment method by a crosslinking agent, polyepoxy compounds. Collagens used in that patent include an insoluble collagen, a soluble collagen, an atelocollagen prepared by removing telopeptides on the collagen molecule terminus using protease other than collagenase, a chemically modified collagen obtained by succinylation or esterification of above-described collagens, a collagen derivative such as gelatin, a polypeptide obtained by hydrolysis of collagen, and a natural collagen present in natural tissue (ureter, blood vessel, pericardium, heart valve, etc.) The Noishiki et al. patent is incorporated herein by reference. “Collagen matrix” in the present invention is collectively used referring to the above-mentioned collagens, collagen species, collagen in natural tissue, and collagen in a biological implant preform.

Sung et al. in U.S. Pat. No. 7,101,857, entire contents of which are incorporated herein by reference, discloses a method for promoting angiogenesis in a subject in need thereof, comprising administering to the subject a substrate loaded with therapeutically effective amount of angiogenesis factor selected from the group consisting of isolated ginsenoside Rg₁, isolated ginsenoside Re or combinations thereof, wherein the substrate is an artificial organ selected from the group consisting of biological patch, cardiac tissue anti-adhesion membrane and myocardial tissue, wherein the substrate is crosslinked with an agent selected from the group consisting of genipin, epoxy compounds, dialdehyde starch, glutaraldehyde, formaldehyde, dimethyl suberimide, carbodiimides, succinimidyls, diisocyanates, acyl azide, tris(hydroxymethyl)phosphine, ascorbate-copper, glucose-lysine and photo-oxidizers.

In one embodiment, the crosslinker or crosslinking agent of the invention may be selected from a group consisting of genipin, its analog, derivatives, and combination thereof, epoxy compounds, dialdehyde starch, glutaraldehyde, formaldehyde, dimethyl suberimidate, carbodiimides, succinimidyls, diisocyanates, acyl azide, tris(hydroxymethyl)phosphine, ascorbate-copper, glucose-lysine, and combinations thereof.

Tissue Specimen Preparation

In one embodiment of the present invention, porcine pericardia procured from a slaughterhouse are used as raw materials. In the laboratory, the pericardia are first gently rinsed with fresh saline to remove excess blood on tissue. The cleaned pericardium before delipidation process is herein coded specimen-A. The procedure used to delipid the porcine pericardia is described below: A portion of the trimmed pericardia is immersed in a hypotonic tris buffer (pH 8.0) containing a protease inhibitor (phenylmethyl-sulfonyl fluoride, 0.35 mg/L) for 24 hours at 4° C. under constant stirring. Subsequently, they are immersed in a 1% solution of Triton X-100 (octylphenoxypolyethoxyethanol; Sigma Chemical, St. Louis, Mo., USA) in tris-buffered salt solution with protease inhibition for 24 hours at 4° C. under constant stirring. Samples then are thoroughly rinsed in Hanks' physiological solution and treated with a diluted cholic acid about 5% at 37° C. for 1 hour. In one embodiment, the cholic acid solution could be from about 2% to about 99%, preferably about 5% to about 50%. This is followed by a further 24-hour extraction with Triton X-100 in tris buffer. Finally, all samples are washed for 48 hours in Hanks' solution and the decellularized sample is coded specimen-B. Light microscopic examination of histological sections from extracted tissue revealed an intact connective tissue matrix with no evidence of cells or cellular residues.

A portion of the decellularized tissue of porcine pericardia (specimen-B) is thereafter lyophilized at about −50° C. for 24 hours, followed by soaking in glycerol-containing fluid (e.g., 75% glycerol and 25% ethanol) to obtain the decellularized dehydrated pericardia. In other experiments, the glycerol content of the glycerol-alcohol mixture may range from about 50 to 100%. In another example, a portion of specimen-B is rinsed and soaked in glycerol-containing fluid (e.g., 80% glycerol and 20% ethanol) to yield decellularized “dry” dehydrated pericardia; optionally, the decellularized dehydrated pericardium is lyophilized at about −50° C. for 24 hours to get a substantially “moisture-free” dehydrated decellularized pericardium. The dehydrated decellularized tissue or pericardial tissue can be re-constituted for medical applications. In a preferred embodiment, the decellularized tissue before lyophilization is thoroughly flushed to remove crosslinking agent, In another preferred embodiment, the decellularized tissue before lyophilization is treated with a counter-agent for a particular crosslinking agent; for example, an amine-containing compound is used to react with the excess free crosslinking agent of epoxy compounds and therefore, deactivate the excess crosslinking agent remained in the tissue.

As disclosed in U.S. Pat. No. 6,998,418, the mechanism of increasing the tissue porosity treated by a mild acidic or base (i.e., a solution pH value greater than 7.0) solution lies in the effect of [H⁺] or [OH⁻] values on the collagen fibers matrix of the decellularized tissue. Similarly, a portion of the decellularized porcine pericardia tissue is further treated with enzymatic collagenase as follows. Add 0.01 gram of collagenase to a beaker of 40 ml TES buffer and incubate the pericardia tissue at 37° C. for 3 hours. The sample is further treated with 10 mM EDTA solution, followed by thorough rinse. In one embodiment, the tissue is stored in phosphate buffered saline (PBS, 0.01M, pH 7.4, Sigma Chemical). In another embodiment, the tissue is lyophilized at about −50° C. for 24 hours, followed by soaking in glycerol to obtain the decellularized dehydrated pericardia. The decellularized dehydrated pericardial patch could be sterilized (for example, EtO sterilization) before use.

Tissue Specimen Crosslinking

The decellularized tissue (specimen-B) of porcine pericardia are fixed with various crosslinking agent. The first specimen is fixed in 0.625% aqueous glutaraldehyde (Merck KGaA, Darmstadt, Germany) as reference. The second specimen is fixed in genipin (Challenge Bioproducts, Taiwan) solution at 37° C. for 3 days. The third specimen is fixed in 4% epoxy solution (ethylene glycol diglycidyl ether) at 37° C. for 3 days. The chemical structure for ethylene glycol diglycidyl ether, one exemplary epoxy compound cited herein, is shown below:

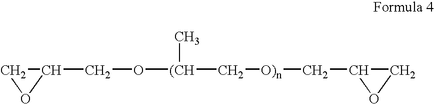

The aqueous glutaraldehyde and genipin used are buffered with phosphate buffered saline (PBS, 0.01M, pH 7.4). The aqueous epoxy solution was buffered with sodium carbonate/sodium bicarbonate (0.21M/0.02M, pH 10.5). The amount of solution used in each fixation was approximately 200 mL for a 10 cm×10 cm porcine pericardium. Subsequently, the fixed decellularized specimens are sterilized in a graded series of ethanol solutions with a gradual increase in concentration from 20 to 75% over a period of 4 hours. Finally, the specimens are thoroughly rinsed in sterilized PBS for approximately 1 day, with solution change several times, and prepared for tissue characterization with respect to degree of crosslinking and appearance. All specimens show crosslinking characteristics per analysis of amino acid residue reactions, increased denaturation temperatures, and resistance against collagenase degradation. The epoxy compounds crosslinked specimen shows whitish translucent appearance with soft flexible feeling; the glutaraldehyde crosslinked specimen shows yellowish appearance with semi-rigid feeling; and the genipin crosslinked specimen shows dark bluish appearance with flexible feeling. The chemical structure for one exemplary genipin cited herein, is shown below:

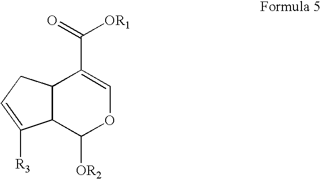

in which
R₁ represents lower alkyl;
R₂ represents lower alkyl, pyridylcarbonyl, benzyl or benzoyl;
R₃ represents formyl, hydroxymethyl, azidomethyl, 1-hydroxyethyl, acetyl, methyl, hydroxy, pyridylcarbonyl, cyclopropyl, aminomethyl substituted or unsubstituted by (1,3-benzodioxolan-5-yl)carbonyl or 3,4,5-trimethoxybenzoyl, 1,3-benzodioxolan-5-yl, ureidomethyl substituted or unsubstituted by 3,4,5-trimethoxyphenyl or 2-chloro-6-methyl-3-pyridyl, thiomethyl substituted or unsubstituted by acetyl or 2-acetylamino2-ethoxycarbonyethyl, oxymethyl substituted or unsubstituted by benzoyl, pyridylcarbonyl or 3,4,5-trimethoxybenzoyl;
provided that R₃ is not methyl formyl, hydroxymethyl, acetyl, methylaminomethyl, acetylthiomethyl, benzoyloxymethyl or pyridylcarbonyloxymethyl when R₁ is methyl, and
its pharmaceutically acceptable salts, or stereoisomers.

In the present invention, the terms “crosslinking”, “fixation”, “chemical modification”, and/or “chemical treatment” for tissue or biological solution are used interchangeably.

Though certain methods for removing cells from cellular tissue and/or acid treatment, base treatment, enzyme treatment to enlarge pores are well known to those who are skilled in the art, it is one object of the present invention to provide a decellularized biological scaffold chemically treated with cholic acid or salts of cholic acid (for example, bile salts) as means of decellularization having increase of porosity for future potential application in tissue regeneration. Some aspects of the invention provide a process for the production of a decellularized pericardial tissue (patch, sheet, strip, and other appropriate shapes or configurations) comprising: (a) providing a pericardium tissue sheet having cells and extracellular matrix; (b) subjecting the sheet to a solution containing bile acid or bile salts which effect the solubilization of cell membranes of the cells present in the tissue sheet; (c) removing the solubilized cell membranes by flushing the tissue sheet with filtered water or other solution; and (d) treating the tissue sheet with a crosslinking agent. In one embodiment, there is provided a process for the production of a decellularized tissue graft by subjecting tissue material (in a non-sheet form) to a solution containing bile acid or bile salts which effect the solubilization of cell membranes of the cells present in the tissue material and optionally treating the tissue material with a crosslinking agent. The bile acid may be cholic acid or its derivatives whereas the bile salts may be glycocholate, deoxycholate, or other cholates.

It is another embodiment of the present invention to provide a tendon or ligament graft for use as connective tissue substitute, the graft being formed from a segment of connective tissue protein or collagen, wherein the segment is decellularized via cholic acid or bile salts and optionally crosslinked. The connective tissue protein may be collagen or pericardia tissue that is substantially devoid of cells adapted for promoting autogenous ingrowth into the graft. The process for using a tissue sheet to make a tendon or ligament graft has been disclosed by Badylak et al. in U.S. Pat. No. 5,573,784, U.S. Pat. No. 5,445,833, U.S. Pat. No. 5,372,821, and U.S. Pat. No. 5,281,422, the entire contents of which are incorporated herein by reference, which disclose a method for promoting the healing and/or regrowth of diseased or damaged tissue structures by surgically repairing such structures with a tissue graft construct prepared from a segment of intestinal submucosal tissue.

Some aspects of the invention relate to a method of repairing a tissue or organ defect in a patient, comprising (a) providing a decellularized tissue sheet material having mechanical strengths; (b) repairing the defect by appropriately placing the tissue material at the defect; and (c) allowing tissue regeneration into the tissue material. By ways of illustration, the tissue sheet material according to the disclosed process of the present invention may be placed at the defect site by suturing, stapling, connecting, or welding to the defect. Other means for placing the tissue sheet material to repair the defect is within the scope of the present invention. In one embodiment, the defect is an abdominal wall defect, a vascular wall defect, a valvular leaflet defect, or a heart tissue defect. In another embodiment, the tissue sheet material further comprises at least one growth factor selected from a group consisting of vascular endothelial growth factor, transforming growth factor-beta, insulin-like growth factor, platelet-derived growth factor, fibroblast growth factor, and combination thereof. In still another embodiment, the tissue sheet material further comprises ginsenoside Rg₁, ginsenoside Re, at least one bioactive agent.

Some aspects of the invention relate to fabrication of a sheet of material that will prevent tissue or organ adhesion post-surgically, to minimize risk of damage from cutting instruments to tissues or organs upon re-operation, comprising: (a) providing a decellularized tissue sheet material produced according to the process of the present invention; (b) placing the decellularized tissue sheet material around, about, or adjacent to the tissue or organ to be treated; and (c) preventing the tissue sheet material from forming the postsurgical adhesion by establishing an anti-adhesion barrier. In a further embodiment, the adhesion is abdominal adhesion. In another further embodiment, the tissue sheet material is crosslinked with a crosslinking agent (for example, epoxy compounds) or with ultraviolet irradiation.

The decellularized pericardial tissue of the present invention is particularly useful as a medical device in orthopedic applications. In one embodiment, the device is used for repair of rotator cuff or strained or ruptured ligaments and tendons. In another embodiment, the device is used as slings for providing proper urethral angle in patients with detrusor dyssynergy that causes urinary stress incontinence. The patients are prone to urinate or void every time they sneeze or dance or do some stressful activity because the support provided by pelvic floor muscle (detrusor weakness) cannot hold the urethra at a proper angle and patient would void against his/her will. In a further embodiment, the device could be used as a membrane for burns or to cover and help the healing of venous or arterial ulcers or diabetes ulcers.

The decellularized pericardial tissue of the present invention is also useful as a medical device to repair chemical burns in the conjunctiva of the eye, to repair vessels large or small, to repair vesicles such as the bladder when torn, or as general surgical reconstruction material. In one embodiment, the pericardial tissue may be used to fabricate or repair tympanic membranes to repair or replace the eardrum, as a fascia lata substitute and possibly other uses. Fascia lata or dura mater could be prepared in the same manner or following the same process of the present invention. The pericardial tissue may be in a form of sheet, patch or strip. The pericardial tissue may also be in a shape of square, circle, rectangle or other configurations.

For tympanic membrane, the material in the raw form can be wrapped around a shape, then fixed with the crosslinking agent while wrapped in the shape such that at completion of fixation it will retain the shape. The shaping instrument can be a mold of a tympanic membrane or the like.

From the foregoing description, it should now be appreciated that a novel and unobvious decellularized pericardium via bile salts and optionally further fixed with a crosslinking agent as a medical device has been disclosed for tissue engineering and medical applications. While the invention has been described with reference to a specific embodiment, the description is illustrative of the invention and is not to be construed as limiting the invention. Various modifications and applications may occur to those who are skilled in the art, without departing from the true spirit and scope of the invention.

What is claimed is:

1. A process for the production of a decellularized tissue, comprising:
providing a tissue sheet having cells and extracellular matrix;
subjecting said sheet to a solution containing bile acid or bile salts that effect the solubilization of cell membranes of the cells present in said tissue sheet;
removing said solubilized cell membranes by flushing the tissue sheet with filtered water or saline; and
treating said tissue sheet with a crosslinking agent.

2. The process of claim 1, wherein the tissue sheet is selected from a group consisting of bovine pericardium, equine pericardium, ovine pericardium, porcine pericardium, caprine pericardium, kangaroo pericardium, fascia lata, and dura mater.

3. The process of claim 1, wherein the crosslinking agent is selected from a group consisting of genipin, epoxy compounds, dialdehyde starch, glutaraldehyde, formaldehyde, dimethyl suberimidate, carbodiimides, succinimidyls, diisocyanates, acyl azide, and combinations thereof.

4. The process of claim 1, wherein the process further comprises increasing porosity of the decellularized tissue.

5. The process of claim 4 in which porosity increase is carried out by a treatment process selected from a group consisting of an enzyme treatment process, an acid treatment process, a base treatment process, and combinations thereof.

6. The process of claim 1, wherein the process further comprises dehydrating said decellularized tissue.

7. The process of claim 1, wherein the process further comprises soaking said decellularized tissue in glycerol or glycerol-alcohol mixture.

8. The process of claim 1, wherein the process further comprises lyophilizing said decellularized tissue.

9. The process of claim 1, wherein the bile acid is cholic acid or deoxycholic acid.

10. The process of claim 1, wherein the bile salts are glycocholate or deoxycholate.

11. A process for the preparation of a decellularized tissue, comprising:
providing a tissue having cells and extracellular matrix;
subjecting said tissue to a solution that effects the solubilization of cell membranes of the cells present in said tissue;
removing said solubilized cell membranes by flushing the tissue with filtered water or saline; and
treating said tissue with a crosslinking agent.

12. The process of claim 11, wherein the solution contains a chemical having a chemical structure with at least two contiguous six-carbon rings shaped in cis-configuration.

13. The process of claim 11, wherein the tissue is selected from a group consisting of bovine pericardium, equine pericardium, ovine pericardium, porcine pericardium, caprine pericardium, kangaroo pericardium, fascia lata, and dura mater.

14. The process of claim 11, wherein the crosslinking agent is selected from a group consisting of genipin, epoxy compounds, dialdehyde starch, glutaraldehyde, formaldehyde, dimethyl suberimidate, carbodiimides, succinimidyls, diisocyanates, acyl azide, and combinations thereof.

15. The process of claim 11, wherein the process further comprises increasing porosity of the decellularized tissue.

16. The process of claim 15 in which porosity increase is carried out by a treatment process selected from a group consisting of an enzyme treatment process, an acid treatment process, a base treatment process, and combinations thereof.

17. The process of claim 11, wherein the process further comprises dehydrating said decellularized tissue.

18. The process of claim 11, wherein the process further comprises soaking said decellularized patch in glycerol or glycerol-alcohol mixture.

19. The process of claim 11, wherein the process further comprises lyophilizing said decellularized tissue.

20. A decellularized pericardial tissue sheet produced by the process in claim 1.
